(19) United States
(12) Patent Application Publication
(10) Pub. No.: US 2004/0127773 A1
(43) Pub. Date: Jul. 1, 2004

(54) Curved rack retractor

(75) Inventor: Peter Douglas, New Milford, NJ (US)

Correspondence Address: SCULLY SCOTT MURPHY & PRESSER, PC, 400 GARDEN CITY PLAZA, GARDEN CITY, NY, 11530

(73) Assignee: Ethicon, Inc., Somerville, NJ

(21) Appl. No.: 10/334,213

(22) Filed: Dec. 31, 2002

(51) Int. Cl. A61B 1/32

(52) U.S. Cl. 600/227

(57) ABSTRACT

A surgical retractor capable of lifting and spreading a surgical opening while minimizing trauma to the patient in a self-contained apparatus. The surgical retractor has a curved rack and two arms. Either or both of the arms is adjustable in position along the rack, and/or configured to traverse the rack, and each arm has a retraction blade. At least one of the retraction blades is configured to securely engage skeletal structure. The retractor rack may also have both a curved portion and a linear portion, with either or both arm adjustable along the curved or linear portion of the rack, or configured to traverse the curved or linear portion of the rack. An accessory arm, articulable with respect to the retractor, may also be provided.

### BACKGROUND OF THE INVENTION

1. Field of Invention

The invention relates to the field of medical instruments, and more particularly, to a curved rack retractor for use in less invasive thoracic surgical procedures.

2. Description of Related Art

Minimally invasive surgical procedures have been developed in recent years to reduce the trauma caused to the patient during surgery. Particularly in cardiac or other thoracic surgeries, the median sternotomy is quite invasive, resulting in increased incidence and/or severity of patient pain and discomfort, complications of surgery, including mortality, and extended recovery periods. One alternative is a thoracotomy procedure.

Thoracotomy procedures use smaller incisions, reducing trauma to the patient. However, in thoracotomy procedures, the surgery is more difficult because of decreased access and visualization of the surgical site. Access is typically provided through an intercostal space that is increased by spreading the ribs with a retractor. Improved access can be obtained with minimal additional trauma to the patient by lifting one of the ribs, typically the superior rib, as it is retracted. Certain apparatus have been devised to perform this lifting and retracting function.

One example is disclosed in U.S. Pat. No. 6,159,231 to Looney, et al. However, the Looney retractor requires an external lifting device to provide lift. A retractor capable of providing lift by its own design is preferable. Another example is disclosed in U.S. Pat. No. 6,416,468 to Deckman, et al. Deckman shows a retractor that lifts without outside apparatus by applying a countering downward force to a footplate on the patient's body. This could also be improved because the downward force of the footplate can cause additional trauma.

### BRIEF SUMMARY OF THE INVENTION

Therefore, it is the object of the present invention to provide a surgical retractor capable of lifting and spreading the ribs of the patient in a self-contained apparatus. It is another object of the present invention to provide a surgical retractor capable of lifting and spreading the ribs of the patient while minimizing additional trauma.

To achieve these and other objects, provided is a surgical retractor comprising a curved rack and a plurality of arms securable to the rack, typically two. At least one of the arms is adjustable in position along the rack, or configured to traverse the rack, and at least two of the arms each have a retraction blade. At least one of the retraction blades is configured to securely engage skeletal structure.

The retractor rack may also have both a curved portion and a linear portion. Moreover, one or more arms may be adjustable in position along the curved or linear portion of the rack, or be configured to traverse the curved or linear portion of the rack. An accessory arm, articulable with respect to the retractor, may also be provided.

### BRIEF DESCRIPTION OF THE DRAWINGS

These and other features, advantages and benefits will be made apparent through the following descriptions and accompanying figures, where like reference numerals refer to the same features across the various drawings.

FIG. 1 shows a perspective view of a curved rack retractor according to a first embodiment of the present invention;

FIG. 2 shows a cross sectional view of the first embodiment in a closed position;

FIG. 3 shows a cross sectional view of the first embodiment in an open position;

FIG. 4 shows a perspective view a curved rack retractor according to an alternate embodiment;

FIG. 5 shows a profile view of a combination linear and curved rack according to another alternate embodiment of the present invention; and

FIG. 6 shows a perspective view of a combination linear and curved rack retractor according to the present invention.

### DETAILED DESCRIPTION OF THE INVENTION

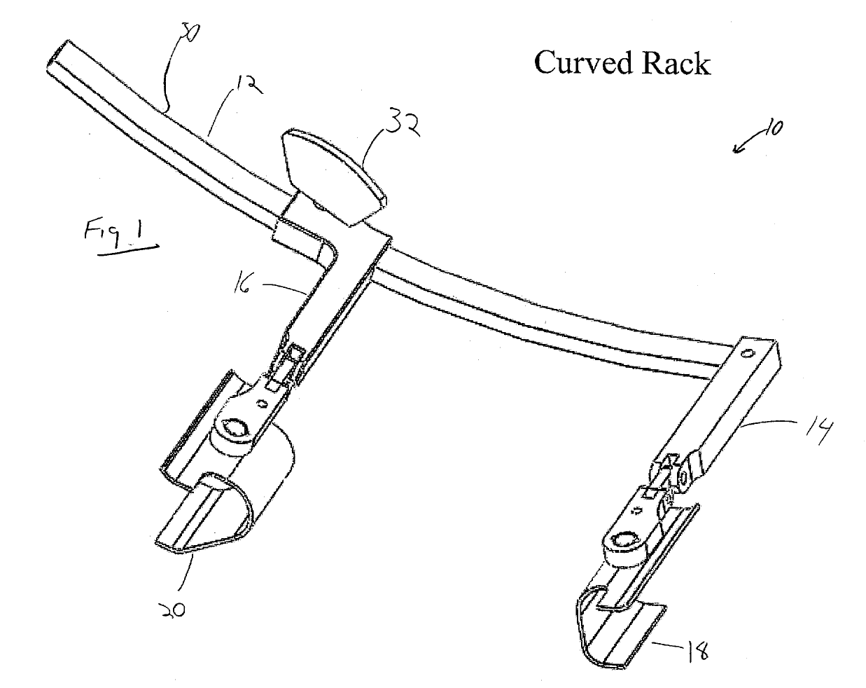

In its preferred embodiments, the present invention is designed for use in less-invasive thoracotomy procedures. With reference to FIG. 1, a first embodiment of the present invention, a surgical retractor, generally 10, has a curved rack 12. FIG. 1 illustrates the rack having a smooth curvature of uniform radius, however the radius of curvature may also vary along the length of the rack. Attached to the rack 12 are a first arm 14 and a second arm 16. The first arm 14 and second arm 16 are provided with inferior and superior retraction blades, 18 and 20, respectively. Second arm 16 is shown to be adjustable in position along the rack 12, with means to traverse the length of the rack 12.

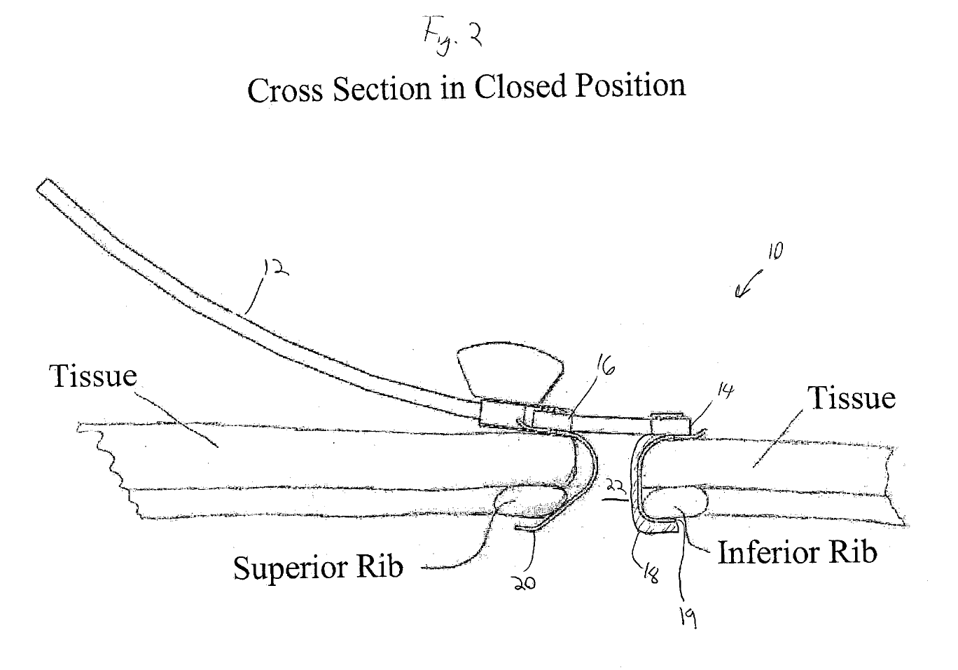

With reference to FIG. 2, a cross section of the retractor 10 in a closed position is shown in use. To use the retractor 10 in a thoracic surgery, the first arm 14 and second arm 16 are brought within proximity of each other. The inferior retraction blade 18 and superior retraction blade 20 are inserted through a surgically formed intercostal space 22, and brought to engage the inferior and superior ribs, respectively.

The inferior retraction blade 18 is configured to anchor the retractor 10 to the inferior rib to prevent rotation around an axis transverse to the rack 12 as the arms are spread. This may be accomplished a number of ways, for example, the inferior retraction blade 18 may be sized to compress the musculoskeletal tissue of the thoracic wall for a secure fit. Alternately or additionally, the inferior retraction blade 18 may include a deformable insert 19, for example made of a cellular foam material, to conform to the shape of the inferior tissue while providing a secure connection. The insert would further reduce the trauma at the attachment point, and/or improve the stability of the attachment. Alternately, an inferior blade 18 may engage the inferior rib, or other skeletal structure, without engaging other tissue. It will be seen in FIG. 2 that inferior retraction blade 18 engages both a skeletal structure, the inferior rib, and the musculoskeletal tissue.

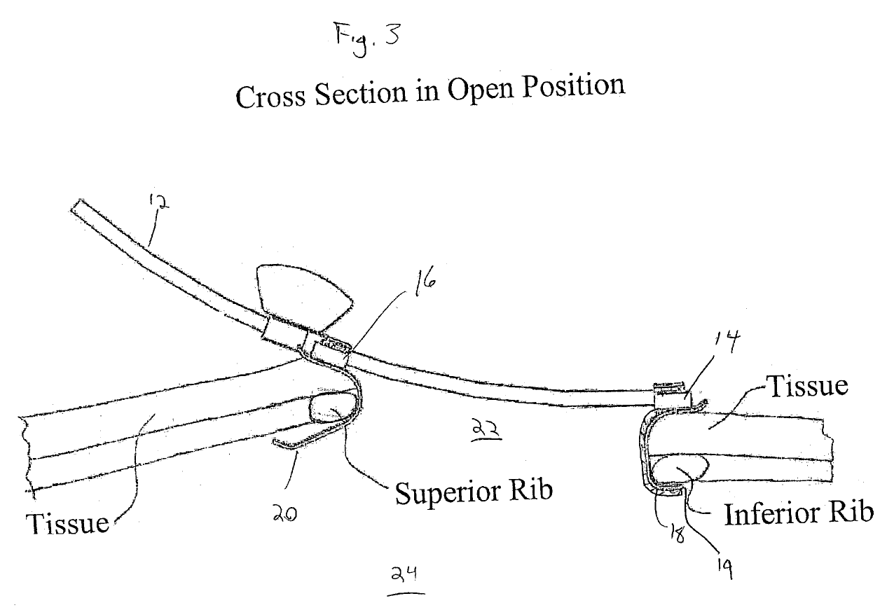

Referring now to FIG. 3, the retractor 10 is shown with the first arm 14 and second arm 16 spread in an open position. The second arm 16 has been moved along the rack 12. The surgically formed intercostal space 22 has been increased to allow surgical access to the thoracic cavity 24 below. By virtue of the curved rack 12, and the engagement of the inferior retraction blade 18 with the inferior rib, the superior rib is lifted as it is spread from the inferior rib, thereby improving access and visualization to the surgical site. This is accomplished without the need for external apparatus to perform the lifting function, or downward pressure on another location of the patient's body.

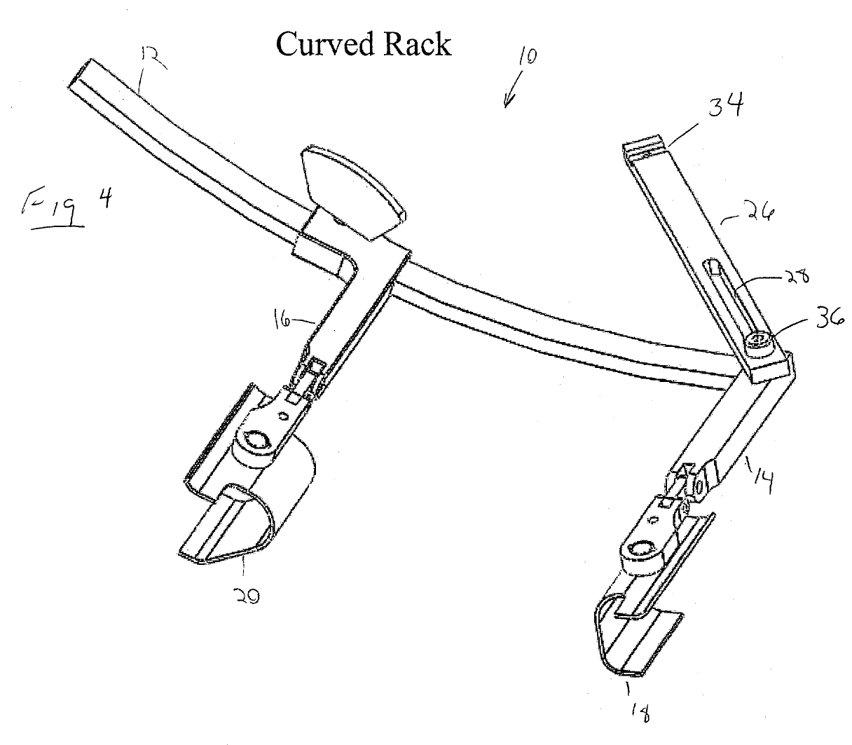

With reference to FIG. 4, a further embodiment of the retractor 10 is shown. In this embodiment, the retractor 10 includes an accessory arm 26. Any of a number of surgical accessories for use during the thoracic procedure may conveniently be secured to the accessory arm 26. One surgical accessory contemplated is an ILLUMINATED VACUUM ASSISTED HEART SURGERY RETRACTOR, which is the subject of co-pending U.S. patent application filed Dec. 19, 2002 by the present inventors, Ser. No. [unassigned] (Attorney Docket No. 16034), hereby incorporated by reference for all purposes. The accessory (not shown) can be secured to the end of the accessory arm 26 at a mounting slot 34, provided for that purpose, by means of a clamp provided on the accessory in a manner known in the art. The accessory arm can include an articulating slot 28 by which it is attached to the rack 12, and through which it may be articulated relative to the retractor 10 to locate any attached surgical accessories.

In use, an accessory will be secured to the accessory arm 26 at mounting slot 30 and located in a desired position by articulation around articulating slot 28. Thereafter, the surgeon secures the accessory arm 26 in the desired position. As illustrated, a thumbscrew 36 is provided to secure the accessory arm 26.

As shown and described in the embodiments of FIGS. 1-4, the first arm 14 has-been fixed to the rack 12. This is most effective when the rack 12 has only a curved portion of constant radius. However, either or both of the first and second arms 14, 16, may be made to adjust in position with respect to the rack 12. In its simplest form, adjusting the position of the arms 14, 16, along the rack 12 includes multiple positions at which the arms may be attached to the rack. After engaging the retraction blades with the respective ribs, the surgeon may manually retract the superior rib, and attach the second arm 16 to the rack 12 at a desired position. More preferably, either or both of first and second arms 14, 16 include means to traverse the length of the rack 12 while attached thereto.

The means to traverse the rack 12 will now be described with respect to the embodiment in which only one arm is adjustable relative to the rack 12. It will be apparent to those skilled in the art that the traversing means described is equally suitable for use on both arms.

One means to traverse includes a rack-and-pinion arrangement. Rack 12 is provided with a toothed area 30 along some or all of its length, but especially over the desired range of motion of the second arm 16. The second arm 16 is also provided with a toothed pinion (not shown) mounted on one end of a pinion shaft (also not shown) and positioned to mesh with the toothed area 30. At the opposite end of the pinion shaft is a key 32 to rotate the pinion shaft and pinion, and thereby adjust the position of the second arm 16.

Additionally, the means to traverse can be provided with means to prevent the unwanted movement of the arm. This can include a ratchet mechanism to prevent rotation of the pinion, or other means as known in the art. Additionally, if a ratchet mechanism is used, a release means may be provided allow free rotation of the pinion, thereby allowing the second arm 16 to move freely along the rack 12.

Other means for adjusting the position of the arms will be apparent to those skilled in the art without departing from the scope of the present invention. Those would include a friction roller in place of the pinion, with a suitable high friction engaging surface in the position of the toothed rack. The friction roller system may also include a ratchet mechanism, and/or a release. Alternately, a cable drive arrangement may be provided to allow the first and second arms 14, 16, to traverse the rack 12. One cable drive arrangement known in the art is disclosed by U.S. Pat. No. 5,944,736 to Taylor, et al., hereby incorporated by reference for all purposes. The cable drive, as illustrated at FIG. 35, includes its own means to prevent the unwanted motion of the arms.

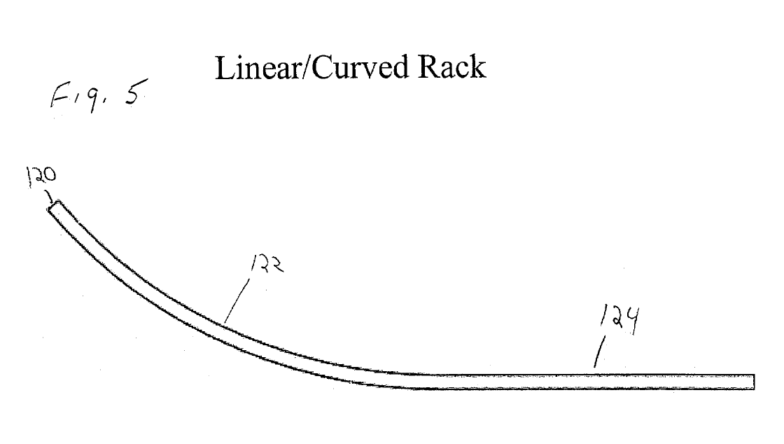

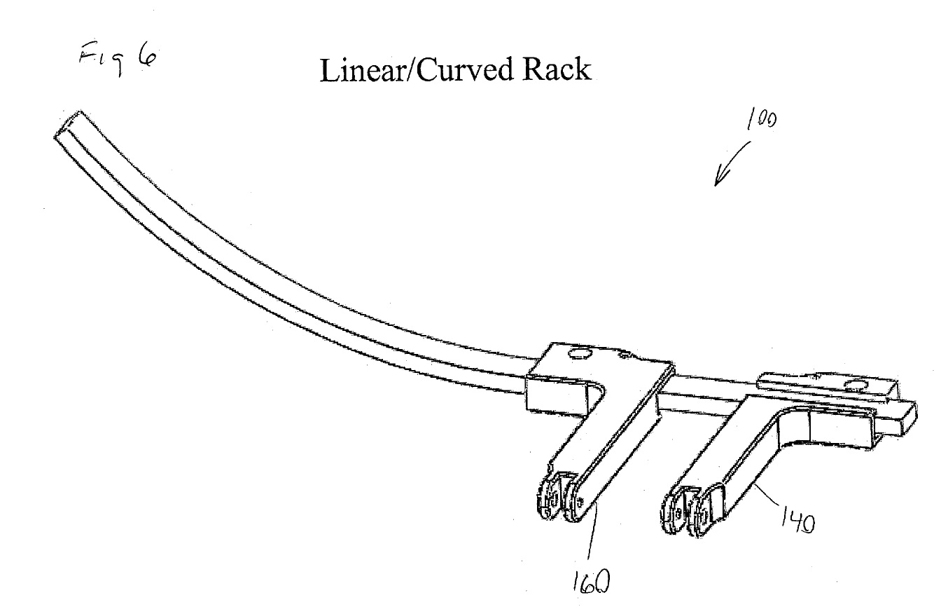

With reference to FIG. 5, shown is a profile view of a rack 120 according to another embodiment of the present invention. Rack 120 has both a curved portion 122 and a linear portion 124. Referring now to FIG. 6, a retractor 100 according to this second embodiment is shown. Retractor 100 includes a first arm 140 and a second arm 160. Preferably, when configured with both a curved portion 122 and a linear portion 124, both first and second arms 140, 160 are adjustable with respect to their position along the rack 120, and/or include means to traverse the rack 120. In this way, the retractor may be used either by positioning the arms 140, 160, along the linear portion, to spread the ribs without lifting, or by using some or all of the curved portion, to provide lift as previously described, or some combination of both linear and curved portions.

The present invention has been described herein with reference to certain preferred embodiments. These embodiments are offered as illustrative, and not limiting, of the scope of the invention. Certain modifications or alterations may be apparent to those skilled in the art without departing from the scope of the invention, which is defined by the appended claims.

1. A surgical retractor comprising:
a rack, the rack having a curved portion;
a plurality of arms securable to the rack, at least one of the arms being adjustable in position along the curved portion of the rack; and
at least two of the arms each having a retraction blade.

2. The surgical retractor according to claim 1, wherein the rack further comprises a linear portion, and at least one of the arms is adjustable in position along at least the linear portion of the rack.

3. The surgical retractor according to claim 1, wherein the rack further comprises a linear portion, and either of two arms is adjustable in position along both the linear portion and the curved portion of the rack.

4. The surgical retractor according to claim 1, further comprising an accessory arm.

5. The surgical retractor according to claim 4, wherein the accessory arm is articulable with respect to the retractor.

6. The surgical retractor according to claim 5, further comprising means to secure the articulable accessory arm in position.

7. The surgical retractor according to claim 4, wherein the accessory arm includes an articulation slot along its length.

8. The surgical retractor according to claim 1, wherein the radius of the curved portion is constant along the length of the curved portion.

9. The surgical retractor according to claim 1, wherein the radius of the curved portion varies along the length of the curved portion.

10. The surgical retractor according to claim 1, wherein at least one adjustable arm further comprises means to traverse the rack while attached to it.

11. The surgical retractor according to claim 10, wherein the means to traverse the rack comprises one or more of a rack-and-pinion, a friction roller, and a cable drive.

12. The surgical retractor according to claim 10, wherein the means to traverse the rack includes means to prevent unwanted movement of the arm.

13. The surgical retractor according to claim 12, wherein the means to prevent unwanted movement of the arm includes one of a ratchet mechanism and a cable mechanism.

14. A surgical retractor comprising:
a rack, the rack having a curved portion;
a plurality of arms securable to the rack, at least one of the arms being adjustable in position along the rack; and
at least two of the arms each having a retraction blade, at least one of the retraction blades is configured to securely engage skeletal structure.

15. The surgical retractor according to claim 14, wherein the retraction blade configured to securely engage skeletal structure is configured to engage musculoskeletal tissue.

16. The surgical retractor according to claim 14, further comprising a deformable insert provided with the retraction blade configured to securely engage skeletal tissue.

17. A method for retracting a tissue wall during surgery comprising:
(a) providing a surgical retractor having:
(i) a rack, the rack having a curved portion;
(ii) a plurality of arms securable to the rack, at least one of the arms being adjustable in position along curved portion of the rack; and
(iii) at least two of the arms each having a retraction blade, at least one of the retraction blades being configured to securely engage skeletal structure;
(b) securing the appropriately configured retraction blade to skeletal structure at an inferior side of a surgical incision;
(c) engaging superior side of the surgical incision with another of the at least two retraction blades; and
(d) adjusting the position of at least one arm along the curved portion of the rack to retract and lift the superior side of the incision.

18. The method according to claim 17, wherein the rack further has a linear portion, and either of two arms is adjustable in position along both the linear portion and the curved portion of the rack.

19. The method according to claim 17, wherein adjusting the position of the at least one adjustable arm comprises traversing the rack.

20. The method according to claim 17, the method further comprising preventing the unwanted movement of the at least one adjustable arm once retracted.
